(19) United States
(12) Patent Application Publication
(10) Pub. No.: US 2025/0370276 A1
(43) Pub. Date: Dec. 4, 2025

(54) MANUFACTURING METHOD OF OPHTHALMIC LENS

(71) Applicant: Shanghai FULO Medical Instrument Co., Ltd., Shanghai (CN)

(72) Inventors: Baoguang JIANG, Shanghai (CN); Mototora KAI, Shanghai (CN); An LI, Shanghai (CN)

(73) Assignee: Shanghai FULO Medical Instrument Co., Ltd., Shanghai (CN)

(21) Appl. No.: 18/814,117

(22) Filed: Aug. 23, 2024

(30) Foreign Application Priority Data
May 31, 2024 (CN) 202410706739.8

(51) Int. Cl. G02C 7/02; B29D 11/00

(52) U.S. Cl. CPC G02C 7/027; B29D 11/00009

(57) ABSTRACT

A manufacturing method of an ophthalmic lens includes the following steps: determining an acquisition area according to a meridional line of an initial lens; randomly selecting from the acquisition area a plurality of initial control points each corresponding to a focal point; determining theoretical axial curvature radii of the initial control points based on the initial control points and the corresponding focal points; determining actual axial curvature radii of the initial control points according to the initial control points; adjusting the initial control points according to a preset condition to obtain target control points respectively. The preset condition includes the actual axial curvature radius of the initial control point being equal to the theoretical axial curvature radius of the initial control point; and manufacturing a lens using all the target control points. The control points are adjusted such that imaging focal points of the control points are changed.

### CROSS-REFERENCE TO RELATED APPLICATIONS

This application claims priority to Chinese Patent Application No. 202410706739.8 with a filing date of May 31, 2024. The content of the aforementioned application, including any intervening amendments thereto, is incorporated herein by reference.

### TECHNICAL FIELD

The present disclosure relates to the field of ophthalmic lenses, and in particular, to a manufacturing method of an ophthalmic lens.

### BACKGROUND

The ophthalmic lens is a medical device for correcting refractive errors such as myopia, presbyopia, and astigmatism of a human eye, including frame glasses, a contact lens, and an intraocular lens. The contact lens further includes a soft corneal contact lens, a hard corneal contact lens, and a scleral contact lens. The intraocular lens further includes a phakic intraocular lens and an aphakic intraocular lens.

In order to increase a focal length of an ophthalmic lens, a bifocus, trifocal, or multifocal optical design is adopted for a traditional ophthalmic lens so that a human eye can see objects at various focal points clearly. However, since the focal points are discontinuous, the vision of a transitional area of the focal points is non-ideal. Therefore, a progressive-focal point optical design is adopted for an existing ophthalmic lens, which provides an optical surface having continuously varying refractive power with a rotational symmetric aspheric surface or a free-form surface, thereby achieving the effect of expanding a focal depth. However, due to its rotational symmetric surface form design, the existing ophthalmic lens cannot correct the refractive error caused by astigmatism. A loop surface design is used for an existing astigmatism correcting ophthalmic lens. Only refractions on two meridional lines are considered. It is possible that refractive correction of a partial area is improper, resulting in visual adverse reaction, such as glare, halo, and double vision.

### SUMMARY OF PRESENT INVENTION

In view of the above shortcomings in the prior art, an objective of the present disclosure is to provide a manufacturing method of an ophthalmic lens. A control point of a spline surface is adjusted such that an imaging focal point of the control point is changed. Focal depth expansion and adjustment are enabled, and a refractive error caused by at least one of myopia, presbyopia, and astigmatism can be corrected.

To achieve the above objective, the present disclosure provides the following technical solutions.

A manufacturing method of an ophthalmic lens includes the following steps:
determining an acquisition area according to a meridional line of an initial lens;
randomly selecting from the acquisition area a plurality of initial control points each corresponding to a focal point;
determining theoretical axial curvature radii of the initial control points based on the initial control points and the corresponding focal points;
determining actual axial curvature radii of the initial control points according to the initial control points;
adjusting each initial control point according to a preset condition to obtain a target control point, where the preset condition includes the actual axial curvature radius of the initial control point being equal to the theoretical axial curvature radius of the initial control point; and
manufacturing a lens using all the target control points.

In a preferred embodiment, the initial lens includes a spline surface; the meridional line of the initial lens passes through a central point of the initial lens; and the determining an acquisition area according to a meridional line of an initial lens includes the following steps:
dividing the meridional line into two half meridional lines symmetrically about the central point of the initial lens; and
determining the spline surface at which the half meridional lines are located as the acquisition area.

In a preferred embodiment, the randomly selecting from the acquisition area a plurality of initial control points each corresponding to a focal point includes the following steps:
selecting the plurality of initial control points from the spline surface at which the half meridional lines are located;
determining a focal length of the half meridional lines from which the plurality of initial control points are selected, where the focal length is in an optical axis passing through the central point of the initial lens; and
setting focal points of the initial control points, where the focal points are in the focal length of the half meridional lines in which the initial control points are located.

In a preferred embodiment, the determining theoretical axial curvature radii of the initial control points based on the initial control points and the corresponding focal points includes the following steps:
establishing a space rectangular coordinate system with the central point of the initial lens as an origin and the optical axis as a Z axis; and
determining respective coordinates of the initial control points and the focal points thereof in the space rectangular coordinate system and calculating the theoretical axial curvature radii of the initial control points.

In a preferred embodiment, the determining respective coordinates of the initial control points and the focal points thereof in the space rectangular coordinate system and calculating the theoretical axial curvature radii of the initial control points include the following steps:
calculating included angles of normals of the initial control points with the optical axis; and
calculating the theoretical axial curvature radii of the initial control points according to the coordinates of the initial control points and the included angles of the normals of the initial control points with the optical axis.

In a preferred embodiment, the determining actual axial curvature radii of the initial control points according to the initial control points includes the following steps:
constructing a spline surface with the initial control points, and calculating the actual axial curvature radii of the initial control points.

In a preferred embodiment, the adjusting each initial control point according to a preset condition includes the following step:
adjusting the initial control point along a direction of an optical axis.

Compared with the prior art, the technical solutions have the following advantages.

A spline surface design principle is introduced into the present disclosure to achieve a focal depth expansion technique. A certain adjusting capability is provided while correcting a refractive error caused by at least one of myopia, presbyopia, and astigmatism. Surface refractive changes are smooth without causing visual adverse reaction. An excellent visual effect is achieved.

A spline surface is used instead of a loop surface and rotational symmetric aspheric surface or free-form surface in the present disclosure. Refractions on more meridional lines are taken into consideration. The probability of improper refractive correction of a partial area when correcting a refractive error caused by astigmatism is minimized.

The present disclosure is wide in application range and can be applied to surfaces of optical bodies of ophthalmic lenses such as frame glasses, a soft corneal contact lens, a hard corneal contact lens, a scleral contact lens, a phakic intraocular lens, and an aphakic intraocular lens.

The following describes the present disclosure with reference to accompanying drawings and embodiments.

### BRIEF DESCRIPTION OF THE DRAWINGS

FIG. 1 is a flowchart of a manufacturing method of an ophthalmic lens according to the present disclosure;

FIG. 2 is a front view of an initial lens according to the present disclosure;

FIG. 3 is an optical path chart of a control point of an initial lens according to the present disclosure;

FIG. 4 is an optical path chart of an initial lens before focal depth expansion according to the present disclosure; and

FIG. 5 is an optical path chart of an initial lens after focal depth expansion according to the present disclosure.

### DETAILED DESCRIPTION OF THE EMBODIMENTS

The following description is used to illustrate the present disclosure such that those skilled in the art can implement the present disclosure. The following preferred embodiments are merely used as an example for description, other apparent variations are likewise conceivable to those skilled in the art. The basic principles of the present disclosure defined in the following description may be applied to other embodiments, variations, improvements, equivalents, and other technical solutions that do not depart from the spirit and scope of the present disclosure.

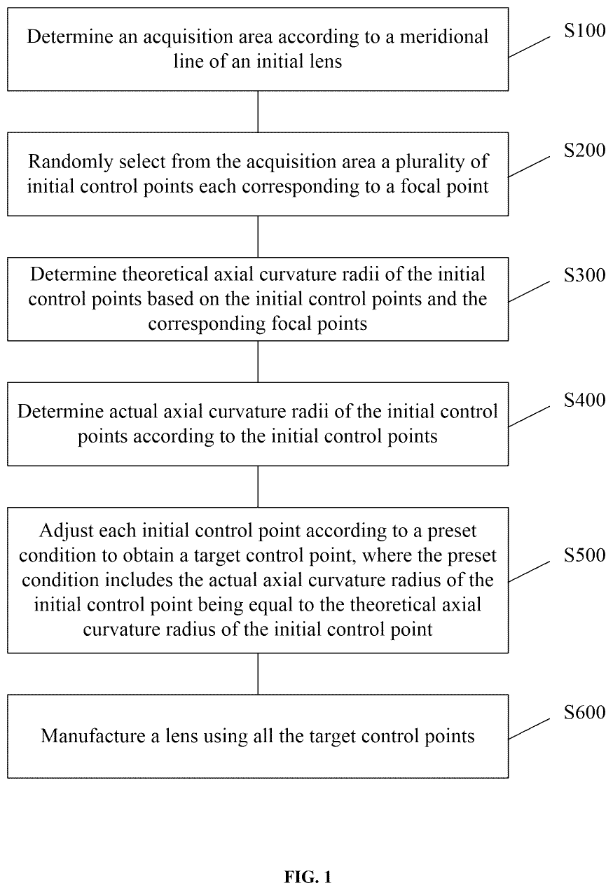

FIG. 1 is a flowchart of a manufacturing method of an ophthalmic lens provided by an embodiment of the present disclosure. The manufacturing method includes the following steps.

An acquisition area is determined according to a meridional line of an initial lens.

A plurality of initial control points each corresponding to a focal point are randomly selected from the acquisition area.

Theoretical axial curvature radii of the initial control points are determined based on the initial control points and the corresponding focal points.

Actual axial curvature radii of the initial control points are determined according to the initial control points.

Each initial control point is adjusted according to a preset condition to obtain a target control point, where the preset condition includes the actual axial curvature radius of the initial control point being equal to the theoretical axial curvature radius of the initial control point.

A lens is manufactured using all the target control points.

An optical surface of the initial lens is a spline surface capable of expanding a focal depth. A control point of the spline surface that is in the meridional line of the initial lens is adjusted, and meanwhile, an imaging focal point of the control point is changed. Focal depth expansion and adjustment are enabled, and a refractive error caused by at least one of myopia, presbyopia, and astigmatism can be corrected.

Specifically, the manufacturing method of an ophthalmic lens includes the following steps.

In step S100, an acquisition area is determined according to a meridional line of an initial lens.

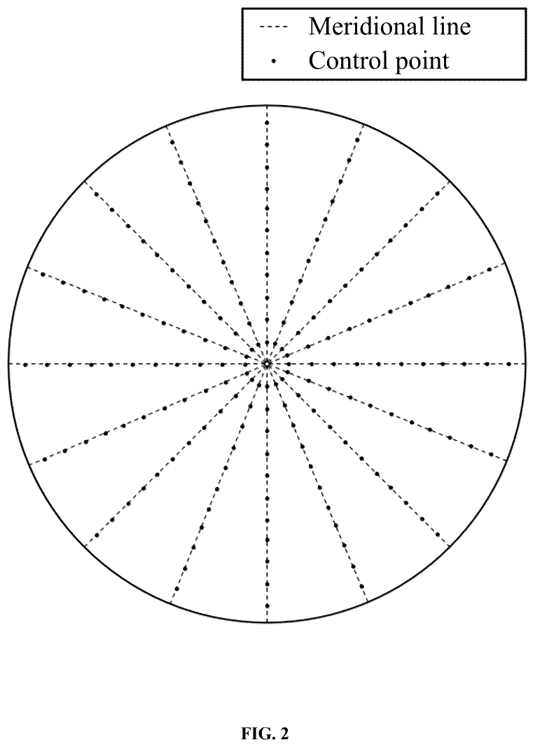

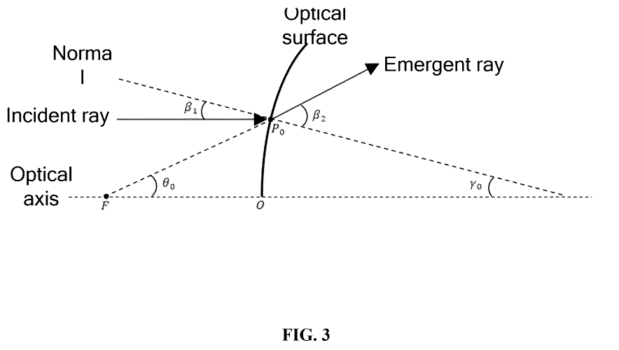

With reference to FIG. 2 and FIG. 3, the initial lens may serve as an optical body, which is closely related to optical phenomena such as propagation, reflection, and refraction of light. An optical surface of the optical body is a spline surface capable of expanding a focal depth. The optical surface may be an internal surface or an external surface of the optical body. The spline surface is composed of a plurality of initial control points. Axial curvature radii of the initial control points are calculated according to preset focal point positions thereof in an optical axis.

With reference to FIG. 2, the initial lens includes a spline surface; the meridional line of the initial lens passes through a central point of the initial lens; and the determining an acquisition area according to a meridional line of an initial lens in step S100 includes the following steps.

In step S110, the meridional line is divided into two half meridional lines symmetrically about the central point of the initial lens.

In step S120, the spline surface at which the half meridional lines are located is determined as the acquisition area.

The optical surface is uniformly divided by a plurality of meridional lines, and the optical surface is the spline surface. The spline surface is composed of a plurality of initial control points, and therefore, the initial control points are present in the meridional lines.

The optical surface is a circular plane having a diameter d, where the optical surface may be uniformly divided by 8 meridional lines. In this case, an included angle between adjacent meridional lines is 22.5°.

In step S200, a plurality of initial control points each corresponding to a focal point are randomly selected from the spline surface at which are half meridional lines are located.

The randomly selecting from the acquisition area a plurality of initial control points each corresponding to a focal point in step S200 includes the following steps.

In step S210, the plurality of initial control points are selected from the acquisition area.

In step S220, a focal length of the half meridional lines from which the plurality of initial control points are selected is determined, where the focal length is in an optical axis passing through the central point of the initial lens.

In step S230, focal points of the initial control points are set, where the focal points are in the focal length of the half meridional lines in which the initial control points are located.

In step S210, with reference to FIG. 1, the acquisition area is defined by the half meridional lines, and each half meridional line may be uniformly provided with 22 initial control points.

In step S220, the focal length is a range defined by a near focal length and a far focal length in the optical axis. The near focal length is close to the central point of the initial lens, and the far focal length is far away from the central point of the initial lens, where the optical axis is a normal of the central point of the initial lens.

The focal length of the half meridional lines is determined according to a correction requirement. In an embodiment, a specific width and position of the focal length are determined according to a specific visual requirement after correction. If the refractive error caused by astigmatism needs to be corrected, the focal length of each half meridional line might be different, and otherwise, be the same.

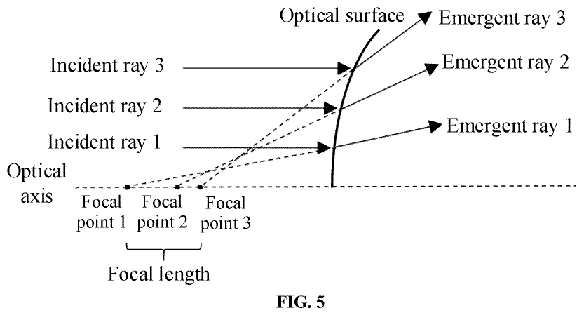

In step S230, a focal point of a control point closer to the inside of the half meridional line is closer to a far focal point. Spacing between focal points of adjacent control points may be equal or may be unequal, or may even be 0, but cannot be all 0. With reference to FIG. 5, in order to expand a focal depth and guarantee a brighter field of view at a near focal point, from the near focal point to the far focal point, the spacing of the adjacent focal points are increasingly greater.

In step S300, theoretical axial curvature radii of the initial control points are determined based on the initial control points and the corresponding focal points.

The theoretical axial curvature radius of each initial control point is calculated according to a positional relationship between each initial control point and the corresponding focal point. Specifically, the determining theoretical axial curvature radii of the initial control points based on the initial control points and the corresponding focal points in step S300 includes the following steps.

In step S310, a space rectangular coordinate system is established with the central point of the initial lens as an origin and the optical axis as a Z axis.

In step S320, respective coordinates of the initial control points and the focal points thereof in the space rectangular coordinate system are determined and the theoretical axial curvature radii of the initial control points are calculated.

In step S310, with reference to FIG. 3, an X axis and a Y axis of the space rectangular coordinate system are set arbitrarily as long as the X axis, the Y axis, and the Z axis are pairwise perpendicular to each other.

The determining respective coordinates of the initial control points and the focal points thereof in the space rectangular coordinate system and calculating the theoretical axial curvature radii of the initial control points in step S320 include the following steps.

In step S321, included angles of normals of the initial control points with the optical axis are calculated.

In step S322, the theoretical axial curvature radii of the initial control points are calculated according to the coordinates of the initial control points and the included angles of the normals of the initial control points with the optical axis.

In step S321, firstly, an included angle of an emergent ray from the initial control point with the optical axis needs to be calculated, and then the included angle of the normal of the initial control point with the optical axis is calculated according to an angle of incidence and an angle of emergence of the initial control point. The specific method is as follows.

In step S3211, P₀ in FIG. 3 is denoted as an initial control point in a spline surface, and its coordinates are (x₀, y₀, z₀).

In step S3212, coordinates of a focal point F of the initial control point P₀ in the optical axis are (0, 0, f₀), and according to a positional geometrical relationship between the initial control point P₀ and the focal point F, the included angle θ₀ between the emergent ray from the initial control point P₀ and the optical axis may be calculated by the following Formula 1.

$${θ}_{0} = arcsin ( \frac{\sqrt{{x}_{0}^{2} + {y}_{0}^{2}}}{{f}_{0} - {z}_{0}} ) . \qquad Formula 1$$

In step S3213, the included angle γ₀ between the normal of the initial control point P₀ and the optical axis is ascertained (as shown in Formula 4) according to a relationship between the angle of incidence and the angle of emergence (as shown in Formula 2) and Snell's law (as shown in Formula 3).

$$\{ \begin{matrix}{β}_{1} = {γ}_{0} \\ {θ}_{0} + {γ}_{0} = {β}_{2}\end{matrix} ; \qquad Formula 2$$

$${n}_{1} sin ( {β}_{1} ) = {n}_{2} sin ( {β}_{2} ) ; \qquad Formula 3$$

$${γ}_{0} = arctan ( \frac{sin {θ}_{0}}{\frac{{n}_{1}}{{n}_{2}} - cos {θ}_{0}} ) ; \qquad Formula 4$$

where β₁ and β₂ represent the angle of incidence and the angle of emergence, respectively, i.e., respective included angles of an incident ray and the emergent ray with the normal of the initial control point; and n₁ and n₂ represent refractive indexes of an incident medium and an emergent medium, respectively.

In step S3214, the theoretical axial curvature radius R₀ of the initial control point may be calculated by the following Formula 5 according to the included angle γ₀ of the normal of the initial control point P₀ with the optical axis.

$${R}_{0} = \frac{\sqrt{{x}_{0}^{2} + {y}_{0}^{2}}}{sin {γ}_{0}} . \qquad Formula 5$$

Optionally, in order to that any focal point within a given focal length has good visual quality, a diopter variation between adjacent control points in the spline surface is not more than 0.5D. If the diopter variation between two adjacent control points is more than 0.5D, a new initial control point is added at a radial midpoint position of two adjacent initial control points, and a focal point thereof is located by default between preset focal points of the two adjacent initial control points.

In step S400, actual axial curvature radii of the initial control points are determined according to the initial control points.

The determining actual axial curvature radii of the initial control points according to the initial control points in step S400 includes the following steps.

A spline surface is constructed with the initial control points according to a B-spline surface equation, and the actual axial curvature radii of the initial control points are calculated.

A B-spline surface is not directly represented by a single equation, but defined by a set of a series of Bezier curves. These curves are joined and interpolated in a certain manner within a parameter space. However, in order to simplify the description, a two-dimensional B-spline surface patch may be considered, which may be defined by the Bezier curves of two parameters (generally denoted as u and v).

It is assumed that there are m control points (P_i,j) in a u direction and n control points in a v direction, where i ranges from 0 to m−1, and j ranges from 0 to n−1. Point P(u,v) in a B-spline surface patch may be then approximated by the following formula:

$$P ( u , v ) = \sum \_ \{ i = 0 \} \hat{} \{ m - 1 \} \sum \_ \{ j = 0 \} \hat{} \{ n - 1 \} B\_i \hat{} m ( u ) * B\_j \hat{} n ( v ) * P\_i , j$$

where B_i∧m(u) and B_j∧n(v) are B-spline basis functions, which define how control points are mixed according to the parameters u and v. The B-spline basis functions are cores of B-spline curves, which are generally defined by a Cox-de Boor recursion formula.

For the B-spline basis function B_i∧m(u), the Cox-de Boor recursion formula is as follows:

$$B\_i \hat{} 0 ( u ) = \{ 1 , if t\_i <= u < t\_ \{ i + 1 \}$$

$$\{ 0 , otherwise$$

$$B\_i \hat{} m ( u ) = ( u - t\_i ) / ( t\_ \{ i + m \} - t\_i ) * B\_i \hat{} \{ m - 1 \} ( u ) + ( t\_ \{ i + m - 1 \} - u ) / ( t\_ \{ i + m + 1 \} - t\_ \{ i + 1 \} ) * B\_ \{ i + 1 \} \hat{} \{ m - 1 \} ( u )$$

where t_i is an element of a knot vector, which defines the parameter space of the B-spline curve. Note that the division operation in the above formula is undefined when the denominator is zero, but in practical use, this may be avoided by selecting a suitable knot vector.

The above formula describes a simple B-spline surface patch. In practical use, a plurality of such surface patches may need to be combined to form a more complex surface. Moreover, the B-spline surface may also be defined and represented in other manners (such as a tensor product and NURBS).

In step S500, each initial control point is adjusted according to a preset condition to obtain a target control point, where the preset condition includes the actual axial curvature radius of the initial control point being equal to the theoretical axial curvature radius of the initial control point.

In step S500, the adjusting each initial control point according to a preset condition includes the following step.

The initial control point is adjusted along a direction of an optical axis.

Specifically, the position of the initial control point is adjusted along the direction of the optical axis using an iterative optimization method (such as a gradient descent method or a genetic algorithm), and steps S300 and S400 are repeated until the actual axial curvature radius and the theoretical axial curvature radius of each initial control point in the spline surface are equal, thereby obtaining the target control point.

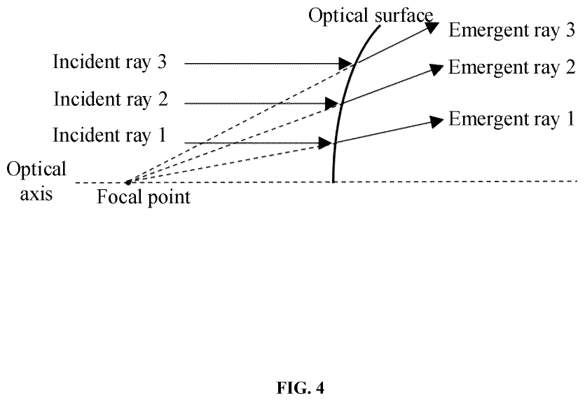

With reference to FIG. 4, the focal points of the initial control points in the half meridional lines are the same, which may be adjusted by the above method steps. With reference to FIG. 5, the focal points of the initial control points in the half meridional lines are different, and in a direction from a near focal point to a far focal point, the spacings of adjacent focal points are increasingly greater, expanding a focal depth and guaranteeing a brighter field of view at the near focal point.

In step S600, a lens is manufactured using all the target control points.

After the coordinates of the target control points are gathered, a dimension parameter of the spline surface may be obtained. The ophthalmic lens can be manufactured according to the dimension parameter of the spline surface.

The ophthalmic lens includes frame glasses, a contact lens, and an intraocular lens. The contact lens includes a soft corneal contact lens, a hard corneal contact lens, and a scleral contact lens. The intraocular lens includes a phakic intraocular lens and an aphakic intraocular lens.

The above embodiments are only used to illustrate the technical ideas and features of the present disclosure, such that those skilled in the art can understand the content of the present disclosure and implement the present disclosure accordingly. The scope of the present disclosure is not limited by the above embodiments, that is, any equivalent changes or modifications made to the spirit disclosed by the present disclosure still fall within the scope of the present disclosure.

What is claimed is:

1. A manufacturing method of an ophthalmic lens, comprising the following steps:
determining an acquisition area according to a meridional line of an initial lens;
randomly selecting from the acquisition area a plurality of initial control points each corresponding to a focal point;
determining theoretical axial curvature radii of the initial control points based on the initial control points and corresponding focal points;
determining actual axial curvature radii of the initial control points according to the initial control points;
adjusting the initial control points according to a preset condition to obtain target control points, wherein the preset condition comprises the actual axial curvature radii of the initial control points being equal to the theoretical axial curvature radii of the initial control points, respectively; and
manufacturing the lens using all the target control points.

2. The manufacturing method according to claim 1, wherein the initial lens comprises a spline surface; the meridional line of the initial lens passes through a central point of the initial lens; and the determining an acquisition area according to a meridional line of an initial lens comprises the following steps:
dividing the meridional line into two half meridional lines symmetrically about the central point of the initial lens; and
determining the spline surface at which the half meridional lines are located as the acquisition area.

3. The manufacturing method according to claim 2, wherein the randomly selecting from the acquisition area a plurality of initial control points each corresponding to a focal point comprises the following steps:
selecting the plurality of initial control points from the spline surface at which the half meridional lines are located;
determining a focal length of the half meridional lines from which the plurality of initial control points are selected, wherein the focal length is in an optical axis passing through the central point of the initial lens; and
setting focal points of the initial control points, wherein the focal points are in the focal length of the half meridional lines in which the initial control points are located.

4. The manufacturing method according to claim 1, wherein the determining theoretical axial curvature radii of the initial control points based on the initial control points and the corresponding focal points comprises the following steps:
establishing a space rectangular coordinate system with the central point of the initial lens as an origin and the optical axis as a Z axis; and
determining respective coordinates of the initial control points and the focal points thereof in the space rectangular coordinate system and calculating the theoretical axial curvature radii of the initial control points.

5. The manufacturing method according to claim 4, wherein the determining respective coordinates of the initial control points and the focal points thereof in the space rectangular coordinate system and calculating the theoretical axial curvature radii of the initial control points comprises the following steps:
calculating included angles of normals of the initial control points with the optical axis; and
calculating the theoretical axial curvature radii of the initial control points according to the coordinates of the initial control points and the included angles of the normals of the initial control points with the optical axis.

6. The manufacturing method according to claim 1, wherein the determining actual axial curvature radii of the initial control points according to the initial control points comprises the following steps:
constructing a spline surface with the initial control points, and calculating the actual axial curvature radii of the initial control points.

7. The manufacturing method according to claim 1, wherein the adjusting the initial control points according to a preset condition comprises the following step:
adjusting the initial control points along a direction of an optical axis.
